(19) United States
(12) Patent Application Publication
(10) Pub. No.: US 2021/0069368 A1
(43) Pub. Date: Mar. 11, 2021

(54) VOLATILE SUBSTANCE DIFFUSER AND METHOD FOR DIFFUING VOLATILE SUBSTANCES

(71) Applicant: ZOBELE HOLDING S.P.A., TRENTO (IT)

(72) Inventors: Moisés CABALLERO TAPIA, Barcelona (ES); Joaquim LLORENTE ALONSO, Barcelona (ES); Sergio LUQUE VERA, Barcelona (ES)

(21) Appl. No.: 16/958,209

(22) PCT Filed: Dec. 27, 2018
(86) PCT No.: PCT/EP2018/097027

§ 371 Date: Jun. 26, 2020

(30) Foreign Application Priority Data
Dec. 28, 2017 (ES) P201731486

(51) Int. Cl. A61L 9/12; A61L 9/14

(52) U.S. Cl. CPC A61L 9/122; A61L 9/14; A61L 2209/133; A61L 2209/111; A61L 2209/12

(57) ABSTRACT

The volatile substance diffuser comprises a housing for a refill (1) containing the volatile substances that are diffused and a motion detector (2) that activates the diffuser upon detecting movement, and it also comprises a timer (3) that activates and/or deactivates a diffuser in accordance with predetermined time periods.

The method comprises the stages of activating the volatile substance diffuser using the motion detector (2); automatically deactivating the volatile substance diffuser after the first predetermined time period has elapsed; and maintaining the volatile substance diffuser deactivated for a second time period.

It provides a diffuser and a method that prevents excessive battery wear and, at the same time, optimises the diffusion rate.

The present invention relates to a volatile substance diffuser, especially designed for use in a vehicle, and to a method for diffusing volatile substances using said diffuser.

### BACKGROUND OF THE INVENTION

The volatile substance diffuser is used in closed spaces such as rooms and vehicles, and these diffusers may comprise or be connected to a battery.

In order to improve its use and increase the duration of the battery or avoid unnecessary costs, volatile substance diffusers comprising motion detectors that only become activated when movement is detected, i.e. the presence of a user, are known.

For example, when the presence of a user is detected by the motion detector a predetermined amount of volatile substances is usually sprayed into the air and the diffuser remains inactive until the presence of a user is newly detected.

In addition to an inadequate use of the battery, this type of diffusers has the drawback of having an inadequate diffusion rate. Thus, if the volatile substances are diffused for a long time continuously, the user becomes accustomed to the fragrance, whereupon it loses its effect. On the contrary, if the volatile substances are diffused at certain times, the diffusion rate is insufficient.

Therefore, an objective of the present invention is to provide a volatile substance diffuser and a method for diffusing volatile substances that will make it possible to avoid excessive use of the battery and, at the same time, optimise the diffusion rate so the user adequately perceives the fragrance of the volatile substances.

### DESCRIPTION OF THE INVENTION

The aforementioned drawbacks are resolved by the volatile substance diffuser and the diffusion method of the invention, and there are other advantages that will be described below.

According to a first aspect, the present invention relates to a volatile substance diffuser comprising a housing for a refill containing the volatile substances that are diffused and a motion detector that activates the diffuser upon detecting movement, and is characterised in that it also comprises a timer that activates and/or deactivates a diffuser in accordance with predetermined time periods.

Due to this characteristic, excessive use of the diffuser battery or battery of the vehicle whereto the diffuser may be connected is avoided. It also prevents the user from becoming accustomed to the fragrance of the substances and not detecting them.

Advantageously, the volatile substance diffuser may also comprise means for regulating the intensity of diffusion of the volatile substances, such that the user can be alerted in this manner, for example, that he/she must stop in case of excessive driving time, since said means for regulating the intensity are preferably connected to said timer, such that they increase the intensity of diffusion of volatile substances after a predetermined time period has elapsed.

The volatile substance diffuser according to the present invention may also comprise a luminous or acoustic indicator, for example, to alert the user when the diffuser is activated or deactivated, and may also comprise an electrical connector, for example, to connect it to a power outlet in a vehicle.

According to a preferred embodiment, said intensity regulation means comprise an electrical resistor, although said regulation can be performed in any adequate manner, for example, by means of a fan.

According to a second aspect, the present invention also relates to a method for diffusing volatile substances using the previously described volatile substance diffuser, which comprises the stages of:
activating the volatile substance diffuser using the motion detector;
automatically deactivating the volatile substance diffuser after a first predetermined time period has elapsed; and
keeping the volatile substance diffuser deactivated for a second time period.

Also due to this characteristic, excessive use of the diffuser battery or battery of the vehicle whereto the diffuser may be connected is avoided. Additionally, it also prevents the user from becoming accustomed to the fragrance of the substances and not detecting them.

Said method may also advantageously comprise the stage of increasing the diffusion of the volatile substances after said first predetermined time period elapses.

If desired, the method may also comprise the stage of increasing the diffusion of the volatile substances at least once during said first time period.

According to a preferred embodiment, said increase in the diffusion of the volatile substances can be performed only once halfway through said first time period.

For example, said first time period may be four hours, and said second time period may be two hours, although this time period may be adjusted in accordance with the envisaged use thereof.

Advantageously, the method according to the present invention also comprises the stage of lighting up a luminous indicator with two different colours, depending on whether it is within the first or second time period, and the colour of the light can change to a third colour when it is within the second half of the first time period.

### BRIEF DESCRIPTION OF THE FIGURES

In order to better understand the foregoing, drawings are provided wherein, schematically and solely by way of non-limiting example, a practical case of embodiment is represented.

FIG. 1 shows an exploded view of the volatile substance diffuser according to the present invention;

FIG. 2 shows a block diagram representing the components of the volatile substance diffuser according to the present invention;

FIG. 3 shows a block diagram representing the method for diffusing volatile substances according to the present invention, according to a first embodiment; and

FIG. 4 shows a block diagram representing the method for diffusing volatile substances according to the present invention, according to a second embodiment.

### DESCRIPTION OF A PREFERRED EMBODIMENT

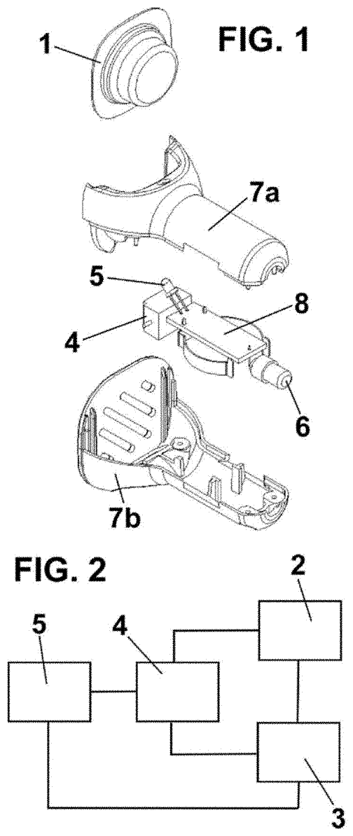

FIG. 1 shows the constituent components of the volatile substance diffuser according to the present invention.

These components are a casing formed by two halves 7a, 7b defining a housing for a refill 1 containing the volatile substances that are diffused into the environment.

Said refill 1 is any conventional refill, which is removed once wasted and is replaced by another.

The diffuser also comprises an electrical connector 6, for example, an electrical connector for vehicles, to feed a resistor 4 that heats said refill 1 for the diffusion of the volatile substances. However, the diffuser may also include its own power supply, for example, one or more batteries.

However, it should be noted that the diffuser may be of any other type without need for using a resistance, for example, using a fan, spraying by means of a valve, nebulisation by means of a generator, etc.

The diffuser may also comprise a printed circuit board 8 whereon the following components are mounted: a visual or acoustic indicator 5, a motion detector 2 and a timer 3.

The visual indicator 5 is preferably a light-emitting diode that can change colour, as will be described below, and the motion detector 2 may be an accelerator, a vibration sensor, an angle sensor, an inclination sensor, a position sensor or any suitable motion detector. The indicator may also be acoustic, emitting a recognisable sound for the user.

The timer 3 may be, for example, a micro-controller or, alternatively, may be any type of electronic component that makes it possible to measure the time and activate and deactivate the diffuser, as will be explained below.

The connection of the electric/electronic components included in the diffuser according to the present invention is shown in the block diagram of FIG. 2.

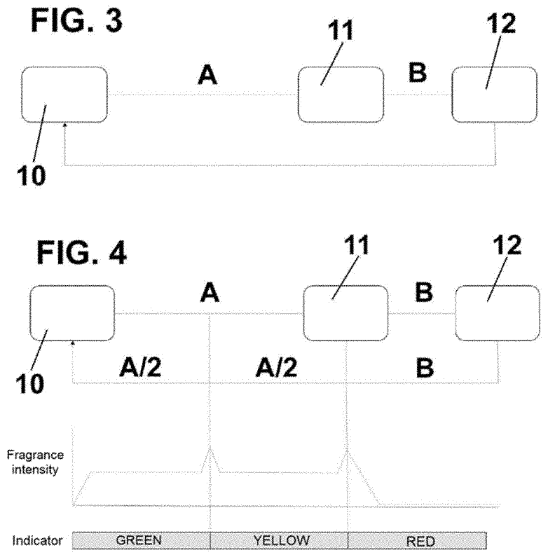

FIGS. 3 and 4 show two methods for diffusing volatile substances according to two preferred embodiments.

According to the first embodiment, in a first stage 10 the volatile substance diffuser is activated when movement is detected by the motion detector 2. At this point, the timer starts counting a first predetermined time period, during which the volatile substance diffuser diffuses said volatile substances.

This first time period is, for example, four hours, although it may be any adequate time period.

If using the diffuser in a vehicle, within the first time period (A), the diffuser is deactivated when the vehicle is not in use, which may be detected by the motion detector 2, to prevent unnecessary use of the battery (included in the diffuser or that of the vehicle).

After said first predetermined time period A has elapsed, in a second stage 11 the timer 3 deactivates the diffuser to prevent volatile substances from being diffused during a second predetermined period B, for example, of two hours. During this second time period B, the motion detector 2 is preferably deactivated as well, since the diffuser will not become activated in any case, even if movement is detected.

This second time period B during which the diffuser is not activated is to prevent the user from becoming accustomed to the fragrance of the volatile substances, in which case the user would not notice its presence and, therefore, the diffuser would not be effective.

After said second predetermined time period B, in a third state 12 the diffuser is reactivated, activating in turn the motion detector 2, whereupon it returns to the first stage 10, performing the diffusion method in cycles.

FIG. 4 shows a second diffusion method according to the present invention.

In this case, the main difference with respect to the first embodiment is, however, in the intensity of the fragrance, maintaining the first, second and third stages in the same manner as in the first embodiment, as explained below.

In this second embodiment, the intensity of the fragrance changes at two moments in time, increasing the diffusion rate of the volatile substances, for example, increasing the intensity of the current fed to the resistor 4 or increasing the speed of rotation of a fan, or similar.

In this manner, after half of the first time period A has elapsed from the first stage 10, for example, two hours, the intensity of the fragrance increases. In the event that the diffuser is used in a vehicle, this may alert the driver that it is advisable to stop and rest.

This increase in fragrance intensity also occurs in the second stage 11, upon deactivating the diffuser, alerting the user of said deactivation.

In the event that the diffuser includes a luminous indicator 5, it may change colour in each time period. Thus, for example, in the first half of the first time period A, the colour of the indicator 5 may be green, changing to orange on reaching the second half of the first time period A, and changing to red during the second time period B. Also, if desired, the indication may be intermittent during some of these time periods, for example, in the second half of the first time period A.

Despite the fact that reference has been made to a specific embodiment of the invention, it is evident for a person skilled in the art that the volatile substance diffuser and the method described are susceptible of many variations and modifications, and that all the aforementioned details may be replaced by other, technically equivalent ones, without falling outside the scope of protection defined by the attached claims.

1. A volatile substance diffuser, comprising:
a housing for a refill containing volatile substances that are diffused;
a motion detector that activates the volatile substance diffuser upon detecting movement; and
a timer that activates and/or deactivates the volatile substance diffuser in accordance with predetermined time periods.

2. The volatile substance diffuser according to claim 1, further comprising means for regulating an intensity of diffusion of the volatile substances.

3. The volatile substance diffuser according to claim 2, wherein said intensity regulating means are connected to said timer, and the intensity of diffusion of the volatile substances is increased after a predetermined time period has elapsed.

4. The volatile substance diffuser according to claim 1, further comprising a luminous or acoustic indicator.

5. The volatile substance diffuser according to claim 1, further comprising an electrical connector.

6. The volatile substance diffuser according to claim 2, wherein said intensity regulating means comprise an electrical resistor or a fan.

7. A method for diffusing volatile substances using the volatile substance diffuser according to claim 1, said method comprising:
activating the volatile substance diffuser upon detecting movement using the motion detector;
automatically deactivating the volatile substance diffuser after a first predetermined time period has elapsed; and
keeping the volatile substance diffuser deactivated for a second predetermined time period.

8. The method according to claim 7, further comprising increasing a diffusion of the volatile substances after said first predetermined time period elapses and before the automatically deactivating.

9. The method according to claim 7, further comprising increasing a diffusion of the volatile substances at least once during said first predetermined time period.

10. The method according to claim 9, wherein said increase in the diffusion of the volatile substances is performed only once halfway through said first predetermined time period.

11. The method according to claim 7, wherein said first predetermined time period is four hours.

12. The method according to claim 7, wherein said second predetermined time period is two hours.

13. The method according to claim 7, further comprising lighting up a luminous indicator with two different colours based on whether the timer indicates the first or second predetermined time period.

14. The method according to claim 13, further comprising changing the luminous indicator to a third colour when the timer indicates a second half of the first predetermined time period.
